(19) United States
(12) Patent Application Publication
(10) Pub. No.: US 2019/0008645 A1
(43) Pub. Date: Jan. 10, 2019

(54) Orthopaedic Implant

(71) Applicant: ZM Praezisionsdentaltechnik GmbH, Rostock (DE)

(72) Inventor: Milija MITROVIC, Rostock (DE)

(21) Appl. No.: 16/066,106

(22) PCT Filed: Dec. 28, 2016
(86) PCT No.: PCT/DE2016/000442

§ 371 Date: Jun. 26, 2018

(30) Foreign Application Priority Data
Dec. 29, 2015 (DE) 10 2015 016 894.0

(51) Int. Cl. A61F 2/32; A61F 2/30; A61L 27/10; A61L 27/06

(52) U.S. Cl. CPC A61F 2/32; A61F 2/30767; A61L 27/30; A61L 27/06; A61L 27/10

(57) ABSTRACT

For an orthopaedic implant, an insert body of a non-metallic material is arranged in a socket of an originally metallic material. The insert body consists of a ceramic based on zirconium dioxide, aluminum oxide or a mixed oxide ceramic, whereas the socket is preferably produced of pure titanium or a titanium alloy, for example Ti-6Al-4V, provided with a plurality of small holes and preferably embodied as a mesh or net structure. The titanium net structure is firstly imparted with ceramic properties with the aid of a silicate glass solder solidified or hardened in a ceramic firing, the subsequent connecting or joining between the ceramic insert body and the now also “ceramic” titanium socket is achieved by means of a glass solder which is based on silicon dioxide (SiO₂) and which connects or joins the two components with one another.

The invention relates to an implant for the replacement of joints, with an insert body of a non-metallic material and a metallic socket that at least partially surrounds this insert body.

Among other things, the implant can involve a portion of a hip joint endoprosthesis, in which a femoral component can be inserted into the long bone, i.e. hollow bone, of the thigh, and in which a cup or shell serves as a sliding tribological partner on the acetabular side. In the latter, an insert body of a non-metallic material is typically arranged in a socket of a metallic material, which provides the contact to the body's own bone tissue.

In the overwhelming majority of the cases today, especially for hip joint endoprostheses, the insert bodies used therein mostly consist of a polyethylene material, which in certain circumstances is highly cross-linked or XPE (cross-linked) polyethylene, and represent one of the most important causes for a loosening of the prosthesis with an inflammation reaction of the joints caused by abrasive wear particles. On the other hand, also with metal/metal sliding tribological pairings or slide bearing couples, for example based on cobalt-chromium or precious metal materials, as are also used, considerable risks arise due to the release of metal ions. In order to largely exclude or prevent such complications, an already known measure proposes to use a non-metallic material as the insert body in an implant of the above mentioned type, thus for example in the already mentioned hip joint endoprostheses, to insert a non-metallic insert body into a metallic socket, which then for example consists of a titanium material. Specifically, in this manner, a good fixing with the surrounding bone tissue can be achieved, for example with a coating of a porous calcium phosphate or titanium applied by a plasma spray method on the outer surface of the socket. In this context, it is also an already known measure to provide such a titanium socket with throughholes.

Ceramics represent an ever-more favored and utilized material for joint implants, wherein, due to their excellent tribologic characteristics, aluminum oxide, zirconium oxide or mixtures of these are frequently used for load-bearing surfaces. Furthermore, these materials are characterized by a good biocompatibility and by a high acceptance in the human body. However, with these materials there generally exists only a relatively low or small tendency toward an osseointegration, and also their flexibility is not the same as with a titanium material. For this reason, a number of surface modifications and coatings have already been proposed for dental and orthopaedic implants, in which a titanium base body is provided with a coating or a surface structuring. In that regard, coatings of zirconium oxide and bioactive glasses are also utilized. In that regard, an especially notable feature is the possibility of a revision operation, i.e., if it should become necessary, an explantation of a previously inserted implant.

The object of the invention is to further develop an implant of the initially mentioned type in such a manner so that on the one hand it connects or joins well with the surrounding bone material in the sense of an osseointegration, and that at the same time the implant also enables a revision operation without difficulties.

The invention achieves this object in that it provides that in such an implant, a ceramic body is surrounded by a socket of a titanium material, wherein the surface of the metallic socket is pre-coated with a silicate ceramic solder that is solidified or hardened by a ceramic firing, and its subsequent soldering with the outer surface of the insert body is achieved via a glass solder based on a silicon dioxide, which joins the two components with one another.

Such an implant represents a biocompatible solution for all joints of the human body, thus, especially for artificial knee, hip-ball, shoulder or fingertip joints; components for the spine, for other small joints also can be produced from the implants according to the invention. In this regard, generative production methods, such as for example the rapid prototyping technique, have been shown to be especially suitable for the production of such implants. The ceramic body inserted into the metallic socket can consist both of a pure aluminum oxide ceramic, a zirconium oxide ceramic or also of a so-called mixed oxide ceramic of aluminum and zirconium oxide, if applicable with an addition of yttrium oxide.

The complete coating of a fully perforated or open-work mesh or net structure of a titanium alloy, which thereby takes on ceramic characteristics, is especially suitable for a cement-free implant with which a secure covering and the capturing of all metal ions that are released out of the joined structure, in this case primarily of titanium oxides, is ensured. In such a fully ceramic mesh or net, the connection with the ceramic body arranged thereunder is produced via a glass solder, which is capable of binding two ceramics with one another. The pre-treatment is carried out by means of a method in which a layer of a ceramic solder is uniformly sprayed on and thereafter subjected to a firing process. Through this firing process, the metal obtains a ceramic-like surface, and not only are all released titanium oxides bound in, but also simultaneously the roughness of the titanium surface is made uniform and therewith an optimal base for the subsequent soldering process is formed.

In the following the invention will be explained in further detail in connection with an example embodiment illustrated in the drawing. It is shown by:

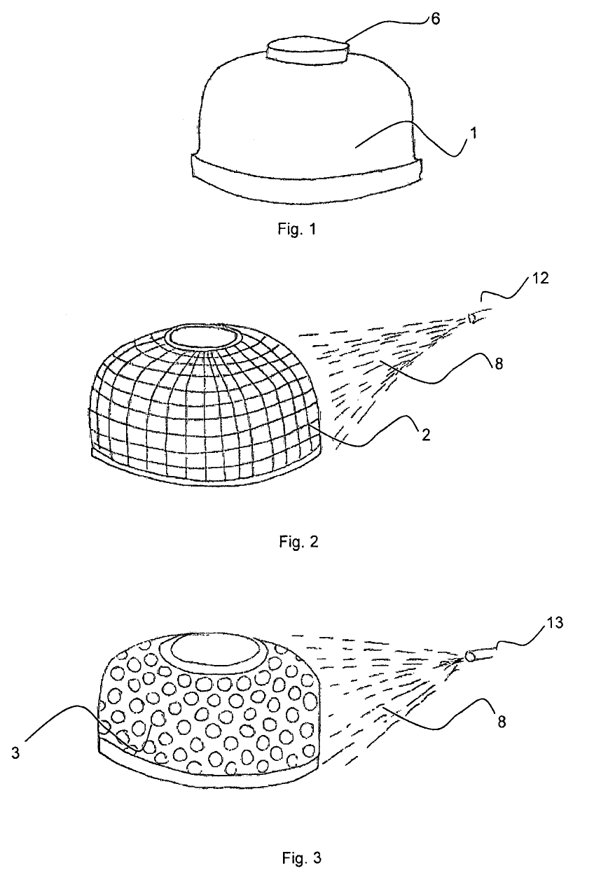

FIG. 1 a ceramic body for a hip joint endoprosthesis in a perspective illustration,

FIGS. 2 and 3 two different embodiments of an originally metallic socket, which surrounds the ceramic insert body, and which is respectively provided with a plurality of small openings,

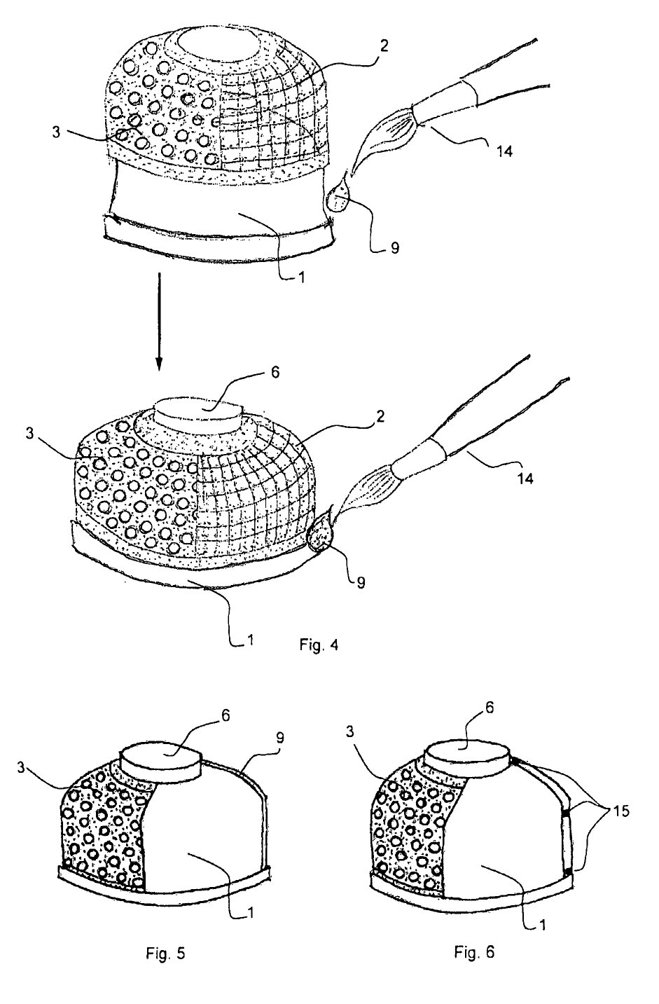

FIG. 4 the joining of a ceramic insert body with the mesh or net structure,

FIG. 5 a schematic illustration of directly allocated socket and insert body, and

FIG. 6 an illustration according to FIG. 5 with spacers between socket and insert body.

The illustration according to FIG. 1 shows a ceramic insert body 1 for a hip joint endoprosthesis, which is inserted in a shell that is embodied originally as a metallic socket or cup 2, 3. The insert body 1 consists of either a pure aluminum oxide ceramic, a zirconium oxide ceramic or of a so-called mixed oxide ceramic of aluminum and zirconium oxide, if applicable with an additive of yttrium oxide, and is provided with a central stopper 6, which is formed on it in a one-piece integral manner, and which extends through a central through-bore of the cup 2, 3, and which, among other things, serves for the flowing-away or discharge of excess solder. In the case of the example embodiments of the invention illustrated here, the material of the cup 2, 3 originally involves the titanium alloy Ti-6Al-4V (titanium grade 5).

In the case of the example embodiments of the invention illustrated in the FIGS. 2 to 4, the titanium cup or shell comprises a plurality of small openings, and can be embodied as a mesh or net structure as illustrated in FIG. 2, or in the manner of a three-dimensional perforated metal sheet 3 according to FIG. 3. By means of an airbrush technique 12, 13 or with the aid of another suitable technique, a thin but covering layer of a silicate glass solder 8 is applied onto the structure, and this is subsequently subjected to a ceramic firing. In that regard it is important that all areas of the titanium cup or shell 2, 3 are coated with the solder 8 and are subjected to a ceramic firing, which then produces a drop-free uniform layer that is solidified or hardened.

Thereafter, the cup or shell 2, 3 is set onto the ceramic insert body 1, either directly or via spacers 14, and is connected or joined therewith via a glass solder 9 based on SiO₂, Al₂O₃, K₂O and Na₂O in a soldering process, in which the temperature is preferably maintained under 850° C. in order to avoid a phase transition of the titanium. Before this soldering process, the webs and the inner rims of the mesh or net structure or the perforated metal sheet structure 2, 3 are coated or covered with the glass solder 9, for example with a paintbrush 14, as this is illustrated in the arrangement according to FIG. 4. In that regard, the spacing distance of the two parts, that is to say of the insert body 1 and of the titanium structure 2 or 3, relative to one another is determined by the layer thickness of the glass solder 9 applied onto the stated parts of the titanium structure 2, 3 and onto the outer surface of the ceramic insert body 1. The connection or joining between the insert body 1 and the structure of the titanium shell 2 or 3 produced by this soldering process is now suitable for a cement-free fixing of the implant in the body of a patient.

The geometry of a sliding tribologic pairing of a knee endoprosthesis is in principle significantly more complex than that of a hip joint, but nonetheless implants of the above described type can be produced and utilized also in this case. Components for the spine, for small joints can also be produced or fabricated of such implants. In each case, it is decisive that the titanium structure at first takes on ceramic characteristics with the aid of a silicate glass solder, so that all metallic abrasive wear particles are surely or reliably enclosed, especially no titanium ions can be released, and later at most silicon wear can be detected, whereby the connection or joining between the ceramic insert body and the similarly “ceramic” titanium socket is achieved by means of a silicate glass solder, which is suitable for connecting or joining both ceramic components with one another.

1. An orthopaedic implant for the replacement of joints, with an insert body of a non-metallic material of a ceramic and with a metallic socket of a titanium material that at least partially surrounds the insert body, characterized in that the surface of the metallic socket (2, 3) is pre-coated with a silicate glass solder that is hardened in a ceramic firing, and its subsequent soldering with the outer surface of the insert body (1) is achieved via a glass solder (9) based on silicon dioxide (SiO₂), which joins the two components with one another, and the metallic socket is embodied as a mesh or net structure (2) or as a perforated metal sheet (3), respectively having a plurality of small holes penetrating therethrough.

2-4. (canceled)

5. The orthopaedic implant according to claim 1, characterized in that the insert body (1) consists of a ceramic based on aluminum oxide ceramic.

6. The orthopaedic implant according to claim 1, characterized in that the insert body (1) consists of a ceramic based on zirconium oxide ceramic.

7. The orthopaedic implant according to claim 1, characterized in that the insert body (1) consists of a ceramic based on mixed oxide ceramic.

8. The orthopaedic implant according to claim 1, characterized in that the metallic socket (2, 3) consists of titanium.

9. The orthopaedic implant according to claim 1, characterized in that the metallic socket (2, 3) consists of a titanium alloy.

10. The orthopaedic implant according to claim 1, spacers (15) provided between the metallic socket (2, 3) and the insert body (1).
